(19) United States
(12) Patent Application Publication
(10) Pub. No.: US 2008/0038755 A1
(43) Pub. Date: Feb. 14, 2008

(54) CELLSPOT APPLICATIONS

(76) Inventors: Lawrence M. KAUVAR, San Francisco, CA (US); William D. Harriman, Alameda, CA (US); Ellen J. Collarini, Oakland, CA (US); April Dutta, Milpitas, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/777,218

(22) Filed: Jul. 12, 2007

Related U.S. Application Data
(60) Provisional application No. 60/830,507, filed on Jul. 12, 2006.
(60) Provisional application No. 60/839,174, filed on Aug. 21, 2006.
(60) Provisional application No. 60/848,112, filed on Sep. 29, 2006.
(60) Provisional application No. 60/911,483, filed on Apr. 12, 2007.

(51) Int. Cl. C12Q 1/70; C12N 5/04; C40B 20/04; G01N 33/53; C12Q 1/68

(52) U.S. Cl. 435/7.32; 435/377; 435/5; 435/6; 435/7.1; 506/4

(57) ABSTRACT

A multiplicity of applications of the CellSpot™ assay method are described. Among these applications are extension to integral membrane protein probes, extension to secretion from bacterial cells, identification of antibodies with enhanced affinity, identification of clones with increased secretion levels, and use of massively parallel screening to identify rare efficacious antibodies.

### CROSS-REFERENCE TO RELATED APPLICATION

This application claims priority from U.S. provisional application 60/830,507 filed 12 Jul. 2006, 60/839,174 filed 21 Aug. 2006, 60/848,112 filed 29 Sep. 2006, and 60/911,483 filed 12 Apr. 2007. The contents of these documents are incorporated herein by reference.

### TECHNICAL FIELD

The invention concerns methods and compositions related to assays of single cells or multiplexed assays of single or multiple cells where microscopic observation is employed to enhance the efficiency of assays involving secreted proteins. More specifically, the invention is directed to improvements in experimental technique, determination of relative affinity of antibodies, parsing of cell populations for desired features, and application of ELISpot techniques to bacterial systems.

### BACKGROUND ART

PCT publication WO 2005/045396 published 19 May 2005 sets forth the work of the present inventors in adapting conventional ELISpot assays to single cell profiling and to improved methods for identifying cells that secrete desired proteins, for example, immunoglobulins of desired specificity using multiplexed forms of this method. The adaptations of ELISpot described in this PCT application can be referred to as CellSpot™ assays. In the ELISpot system, a surface, typically a microtiter plate is coated with a capture reagent, typically an antibody for, for example, a cytokine, which is secreted. Cells suspected to secrete the protein are incubated in the wells for sufficient time to permit secretion to occur. After the cells are washed out of the wells, any secreted protein bound to the capture antibody is detected.

The above-cited PCT publication describes how such assays can be multiplexed by using not a single capture reagent, but a multiplicity of capture reagents for different secreted proteins on a capture surface as well as by distinguishing individual proteins by applying uniquely labeled particulates that can be detected individually employing a microscope. Even when a single capture reagent is used, the uniquely labeled particulates can be used to discriminate among the cells producing co-captured proteins, e.g., immunoglobulins. Further, the above cited PCT publication describes detection of secreted proteins from individual cells and evaluating the level of secretion of individual cells by observing the number of particulate labels associated with a secretion footprint from the individual cells. As further disclosed in this publication, individual cells having desirable footprints can be recovered and cultured when a desired footprint is obtained, as the cells can be supported on a membrane which can be removed following capture of secreted proteins so as to permit the assay to be conducted. The location of the secretory cell is then correlated with the location of the footprint on the capture surface. This permits culture and further work on the desired cells.

A somewhat different approach to large-scale testing as compared to that described in WO 2005/045396 has been published recently: Love, J. C., et al., Nature Biotech. (2006) 24:703-707. This approach is based on microlithography to create small wells into which hybridoma cells are deposited, thereby miniaturizing the 96-well plate ELISA format. The secreted antibody is obtained by sampling the supernatant via inverting the array onto a glass slide.

In practice, the cells are not clonal in this format. As described, 50-75% of the wells get 1-3 cells, and only 17 of 50 picked wells were confirmed positives after subcloning, consistent with lack of clonality. This approach would be impractical when the cell density is sufficiently reduced to assure clonality. In addition, as there is no replica of the cell, any picked cell must grow in order not to be lost. In contrast, the CellSpot™ assay described in the above-referenced PCT publication permits 2-3 orders of magnitude higher density with assurance of clonality. In addition, the above-described CellSpot™ assays require less time to obtain results, since sampling of supernatants is not required, and is more reproducible than replicate sampling of microlithography wells.

The present application describes new applications and improvements with respect to these techniques.

### DISCLOSURE OF THE INVENTION

In a one aspect, the invention is directed to a method for obtaining antibodies immunoreactive with a particular desired epitope of a target protein. Antibodies to this particular epitope may need to be obtained in order to produce a desired functional effect. Instances are known where antibodies raised with respect to, for example, a receptor protein are able to bind the receptor, but do not inhibit its activity. In such instances, the antibodies raised by immunization with the full-length protein do not bind to an appropriate epitope so as to interfere with activity, possibly because the target region is not sufficiently immunogenic. Because the method of the invention provides the opportunity quickly to screen a multiplicity of candidate antibodies for multiple traits, multiple individual fragments of the protein can be employed to raise antibodies, with enhancement of immunogenicity by coupling to, for example, tetanus toxoid or keyhole limpet hemocyanin (KLH) or other immunogenicity-boosting components. This permits antibodies to be raised to every region of the target protein. Thus, for example, a receptor protein could be divided into 5, 10, 15 or 20 or more individual peptide fragments, each coupled to an immunogenicity-enhancing agent and used for immunization. The techniques of the present invention can then be used to identify cells that secrete antibody immunoreactive with each region of the target protein. The cells identified as secreting antibodies that meet the criteria of reactivity with the fragment and the intact protein, but not with the remaining fragments, can then be tested for the desired functional activity with respect to the target. The several orders of magnitude increase in efficiency with which immunoglobulin-secreting cells can be screened using the invention methods, as compared to standard hybridoma screening, makes this approach practical.

In another aspect, the invention is directed to a method to culture bacteria so that proteins secreted by the bacteria, or secreted into the periplasmic space, or otherwise released from cells, can be captured on a surface to permit imaging using microscopic techniques. Standard culturing techniques suitable for eukaryotic cells as previously disclosed are not successful using bacteria. In the invention method, aerobic bacteria, such as E. coli, are grown on a porous membrane at sufficient dilution to provide distinct colonies derived from single cells. The pores in the membrane are sufficiently small to prevent passage of the bacterial cells, but of sufficient size to permit proteins and small molecules to be passed, so that nutrients can be supplied from beneath this membrane, and secreted proteins can likewise transit the membrane. Placed under the membrane is a capture surface for imaging. The capture surface permits transit of nutrients supplied from below. An important feature of the capture surface is that it provides a flat surface onto which capture reagent can be coupled. A preferred example is nuclear track etched polycarbonate. Thus, the particulate labels, when they are not bound to the capture surface, may be washed out at the appropriate time as the particulate labels do not embed into the capture surface as would be the case for a more fibrous membrane, but remain, when bound, only at the surface to provide a single focal plane for microscopic observation. A nutrient agar may be placed below the capture surface as a convenient way to supply nutrients to the bacteria. The capture surface will also include capture reagents for the secreted proteins to be detected, if desired. Sufficient leakage occurs from the periplasm in the case of E. coli to permit capture and detection of secreted proteins at the capture surface, even from colonies that are microscopic in size. Alternative methods of protein release include: viral or chemical lysis of cells, proteins attached to budded viruses or simple leakage of intracellular contents. Viral release from mammalian cells is similarly detectable.

It is important, in many contexts, to quantify the number of virus particles present in a specimen. One approach is to use an ELISA assay with an antibody directed at a viral antigen. Such assays often have too high a noise level to be practical for measuring low virus counts, and they do not distinguish between viable infectious virus and damaged or defective virions. The most reliable and sensitive assay is a plaque assay, in which a lawn of susceptible cells is exposed to the specimen and the number of infectious virus particles quantified as the number of plaque forming units (pfu); however, a typical pfu determination requires rounds of virus growth and infection of nearby cells (after lysis of the originally infected cell or after budding out of virus). The resulting cellular debris thereby becomes directly visible, or there is enough virus protein to be detectable by immunohistochemistry. CellSpot™ assay, with or without a membrane to support the cellular lawn, offers a high sensitivity assay for virus production and release from the cells (either by lysis or budding).

The CellSpot™ system is also particularly useful in screening for desired combinations of heavy and light chains. These can readily be produced by E. coli and assembled in the periplasm. By introducing a set of 10, 20, 50 or 100 light chain encoding sequences and 10, 20, 50 or 100 heavy chain encoding sequences into a population of bacteria, combinations of heavy plus light chain assemblies can be constructed that number as the product of the numbers of each. As individual micro-colonies can be monitored using this system, the screening of sufficient cells to assess these combinations is possible.

In one application of this method, E. coli are modified to express the heavy and light chain portions of human Fab fragments and plated at individual cell dilution levels on a nitrocellulose membrane, which serves as the porous membrane. The capture surface situated below includes, for example, polyclonal goat antihuman antibody as capture reagent. Nutrient supply agar is placed under the capture surface which is sufficiently porous to permit nutrients to pass. After sufficient time has elapsed for growth of the single bacteria into small colonies, with secretion of the Fab proteins into the periplasm and subsequent leakage into the media and thence through the supporting membrane to the capture surface, the supporting membrane and nutrient supply portions of the assembly are removed and the capture surface is treated with particulate labels to detect the captured Fab units. As before, labels of multiple different colors are used. In this case, a first color (e.g., red) is associated with particulate labels to which are coupled an antigen specific for the desired Fab protein and particulate labels of different colors (e.g., green, purple, orange) are coupled to other antigens to which the desired Fab should not bind. Imaging is possible under both high and low magnification and in both color channels.

Under low resolution (1.6×) colony secretion footprint diameters of 0.1-1 mm are observed after overnight culture at 30° C. and under high magnification images (40×) individual particles are resolvable in both color channels. A preponderance of red labels indicates Fab protein of the desired specificity is secreted by the cell.

In another aspect, the invention is directed to methods to employ epitopes of membrane-bound proteins as detection reagents for secreted antibodies or other secreted proteins. In some cases, such membrane-bound proteins have sufficient extra-cellular portions available for use as soluble reagents coupled to detection beads. Alternatively if sufficient extracellular portions are exposed at the surface they can be used directly, while still alive, as capture reagents for secreted proteins. After capture, the cells can then be fixed and stained and labeled with particulate labels appropriate to the secreted protein, including simple binding or stimulation of a signal transduction pathway.

In another approach, if the membrane-bound protein can survive disruption of the cell, the freed membrane-bound protein can be coupled to particulate labels by means of a moiety on said particulate labels complementary to a binding partner on the intracellular portion, for example, a capture antibody to an epitope on the intracellular portion. To ensure that such an epitope exists, an added intracellular portion can be fused genetically to the protein, such that this added portion can be matched to a capture reagent (i.e., a moiety complementary to said binding partner) on the particulate labels. These added intracellular regions can include a fusion tag—for example, histidine tags, FLAG label, or an enzyme with a compatible suicide substrate for covalent attachment to the particulate label. One such fusion tag is, for example, the commercially available Covalys SnapTag system, where the complementary moiety is a suicide substrate for the enzyme that constitutes the binding partner. As noted, the counterpart capture reagent to the intracellular extension is coupled to the particulate label and the association of the membrane-bound protein to the particulate label is effected by association with its corresponding partner. If the native intracellular portion can be matched to a complementary moiety, addition of heterologous fusion tag is not necessary.

As an alternative to recombinant production in host cells, and assuming posttranslational modifications are not required, the membrane-bound protein may be produced in a cell-free system in the presence of a suitable detergent. Isolated membrane-bound ribosomes associated with eukaryotic or prokaryotic cells are mixed with the appropriate tRNA, ATP, and amino acids and synthesis is effected by addition of mRNA encoding the membrane-bound protein, said protein optionally including a fused tag. The resulting protein is then solubilized by the detergent, and can then be coupled to the particulate labels as described above.

As noted above, if the membrane-bound protein does not survive cellular disruption, another approach may be used to solve the problem of using such protein as a protein detection ligand for scanning the output of a field of secreting cells, either bacterial or eukaryotic as described above. In some cases, two capture populations of cells are supplied to the capture surface. One population expresses the membrane-bound protein at a high level, and the other population at a low level or not at all. The first and second populations are differentially stained, and may either be alive or dead. The cells may be fixed, for example, with methanol/formaldehyde. The staining is accomplished, for example, by incubating one population with a fluorescent DNA-intercalating dye or any other stain. Similar procedures are used to stain the other population of cells, but using a dye of a different color. The stained cells are then applied to a capture surface of, for example, an ELISpot or CellSpot™ type assay assembly.

In one embodiment, protein-secreting cells plated at appropriate dilution on a supporting membrane are superimposed on the capture surface placed beneath it and incubated for sufficient time to secrete a desired protein, such as an immunoglobulin. After removal of the membrane supporting the protein-secreting cells, any unbound secreted protein is washed from the capture surface and the capture surface is treated with any suitable detection label coupled to a binding partner for the secreted protein. The association of the detection label with the population of cells that express the membrane-bound ligand, but not with the second population that does not express the membrane-bound ligand at high levels, can be confirmed by observing the association of the labels with the color generated by the population of cells producing membrane-bound ligand, but not with the color associated with the population of cells that do not produce it. Because the cells are smaller in diameter than the field of observation, multiple individual cells of both types can be present in the secreted protein “footprint” of a single secreting cell or micro-colony. By appropriate image registration, the position of the secreting cells on the supporting membrane may be correlated with the positions of reactive cells on the capture surface.

Alternatively, the two cell populations can be used in separate wells with the protein secreting cells replicated on the two surfaces. In an alternative detection method, intracellular signaling can be used to visualize binding of the secreted protein. In this embodiment, the capture surface is coated with living cells, and after the appropriate time for secretion, the capture surface is removed and treated to fix the cells. The fixed cells are then made permeable and stained appropriately for the results of intracellular signaling.

In still another aspect, the invention relates to improvements in the CellSpot™ system described in the above-cited PCT publication. Several of these improvements relate to verifying the position of footprints on the capture surface relative to the cell generating the footprint on the superimposed membrane (i.e., improved image registration). Because the membrane that contains the cells needs to be removed before the footprint can be assayed, the membrane needs to be repositioned with respect to the capture surface once the assay has been accomplished so that the appropriate cells can be removed for further study. Several independent improvements make possible more accurate repositioning. These include 1) introduction of a grid pasted to the bottom of a microplate carrier that holds the membrane on which the cells are positioned, which compensates for variable optical aberration caused by the viscous cell immobilization medium; 2) scattering relatively large fluorescent particles (5-10 μm diameter) onto the cell-containing membrane along with the cells to provide a pattern that can be recorded before removing the membrane from the footprint surface, thereby allowing fine scale image registration by matching local geometry of these landmark beads; 3) an improved cell immobilization medium, Mebiol® Gel; and 4) a means for sliding the stage holding the cell membrane laterally from under the microscope to permit vertical access by pipette. Mebiol® Gel is a lipophilic synthetic polymer that has a fine mesh structure at the molecular level and has the characteristic that it is liquid at low temperature but gels upon warming. It is commercially available. Since the cells can grow in Mebiol during analysis of the secreted protein in the CellSpot™ assay, further software improvements allow registration of the center of the microcolony with the originating single progenitor cell.

Another aspect of the invention is an improved method to immortalize human peripheral blood cells, specifically to provide them in a condition for application of the CellSpot™ method of the invention by harvesting them before macroscopic cultures are obtained. The standard method of providing immortalized cells that secrete immunoglobulins is the production of hybridomas through fusion of antibody-secreting cells with tumor cell lines. Alternatively antibody secretion can be enhanced by stimulating with a non-specific mitogen, such as pokeweed. The present invention method comprises infecting the cells with Epstein-Barr virus (EBV) and harvesting the cells after only 10-20 copies are obtained. The advantage of this method is that a substantial proportion of all resting B lymphocytes can be induced to proliferate and secrete immunoglobulin, albeit transiently. Since the CellSpot™ method is so sensitive, the limited number of divisions required following EBV transformation before assay yields satisfactory immunoglobulin-secreting cells. In one application of this method, groups of 10-1,000 parental cells are transformed with EBV and cultured until 10-20 copies are obtained. The resulting population is divided into two portions, one of which is assayed for production of the desired immunoglobulin, and the other which is reserved. If the assay portion of cells give evidence that cells that secrete the desired immunoglobulin are present, the reserved portion of cells can be used as a source for identifying individual members of the population that successfully secrete immunoglobulin. If the assay portion of the cells shows no evidence that it contains cells with the desired secretion characteristics, the reserved portion need not further be addressed.

Still another aspect of the invention permits identification of antibodies that have high affinity for the desired antigen. In the previously published description of CellSpot™ referenced above, a method was disclosed for normalizing for cell number and overall protein concentration by providing a single cell assay and by utilizing particulate labels of varying specificity—one label coupled to a protein reactive with immunoglobulins generally and distinguishable in hue from other particulate labels coupled to antigen(s) for which the antibody specificity is desired. While correcting for different amounts of immunoglobulin present in a particular CellSpot™, these methods, however, did not distinguish between the affinity of binding of an individual antibody/antigen combination and avidity—i.e., enhanced binding due to multiple interactions between the binding partners. The influence of avidity is endemic with respect to the particulate labels, since a multiplicity of antigen copies is displayed on each particle.

In the present invention, avidity is controlled by suitable spacing of the capture reagent on the capture surface. By varying the densities of diluted and spaced capture reagent, high affinity clones can be distinguished from those with low affinity by virtue of the retention of the ability of the capture reagent to bind secreted antibody even at very low capture reagent density. Affinity ranking as determined in this manner correlates with assessment using the Biacore™ or other high precision methods. While the foregoing method is particularly conveniently conducted using the CellSpot™ technique, this is not a requirement, and any means of applying the protein to the capture surface, such as treating the surface with a solution of the protein is satisfactory. Further, although the foregoing method is illustrated using immunoglobulins as an example, any binding partner interaction can be explored in this manner. Thus, the affinity of a ligand for its receptor, for example, could be determined in this manner, as compared to known standards, as could the affinity of various fusion tags for the their complementary moieties. In another application, the degree of homology of nucleotide sequences can be at least qualitatively determined.

In still another embodiment, the invention is directed to the use of the CellSpot™ method for identifying cells with high levels of secretion of a desired protein, for example relative to insertion into sites that lead to such high expression levels of desired proteins. In this illustrated method, the nucleotide sequence encoding a desired protein is randomly cloned into a population of cells and each individual cell, or its clonal progeny, is evaluated for the level of secretion. Secretion levels are readily determinable by the intensity and/or diameter of the footprint of single cells with respect to the expressed protein, as previously disclosed. Because of the high throughput nature of the CellSpot™ assays, many insertion sites can be evaluated efficiently and the highest secreting cells recovered and cultured, which is useful in selecting for a manufacturing cell line. The insertion site in the recovered cells can also be determined by genetic analysis, enabling subsequent direct targeting to a particularly favorable site.

This method may also be used to evaluate the effect of different growth medium formulas on secretion levels, and simply to evaluate secretion levels per se. Similarly, stability of expression using clonal expansion is monitored. Thus, the determinations are made as a function of time.

Another aspect of the invention relates to an efficient method to identify cells that provide high levels of secretion of one or more proteins or that secrete protein of the correct specificity by a process designated “binning.” In this process, a multiplicity of cells is tested simultaneously for desired secretion characteristics by assessing footprints of secreted proteins left by each individual cell in a “bin” of sufficient size and configuration that individual footprints can be discerned and associated with individual cells for a multiplicity of individual cells contained therein. By assessing a multiplicity of individual cells simultaneously, collections that contain high numbers of high secreting or appropriate cells can be used as a source for such cells, which can be identified individually by the methods of the invention.

Pooling signal from multiple cells in prior art methods masks the presence of favorable outliers due to dilution of the signal from that cell. In conventional methods, the only way to avoid this averaging effect is to clone the cells before assay. Because CellSpot™ has the sensitivity to read the secreted protein from single cells, a multiclonal bin can be assessed at single cell resolution, reserving the labor intensive and time consuming cloning step for the small fraction of bins that contain favorable cells.

In all of the above methods, the CellSpot™ method is useful to increase the number of cells it is possible to examine by several orders of magnitude as compared to conventional methods based on limiting dilution cloning prior to assay, thus permitting selection of rare cells that provide secreted proteins with particularly favorable traits.

In another aspect, the invention relates to a method to screen very small quantities of members of a combinatorial library (composed of small molecule compounds or larger biological products) which method comprises applying the members of the library to a capture surface and treating said surface with detectable forms of desired binding partners. The desired binding partners may be antibodies, for example, or recombinantly produced cell surface receptors, receptor ligands, and the like. As each individual position on the array can be interrogated, the ability of the individual member of the library to bind the potential binding partner can be determined.

Alternatively, the capture surface may comprise the desired binding partner and a multiplicity of members of a combinatorial library, each labeled with a distinctive label used to interrogate the surface.

In still another aspect, the invention is directed to a method to detect endocytosis by assessing nuclear fluorescence generated by an intercalated dye borne by the endocytosed or internalized substance.

The benefits of multiplexed probing of microscopic quantities of analyte are illustrated herein using antibodies as binding agents to antibodies. The same benefits apply to other recombinant proteins and peptides. Further, the same benefits apply to synthetic chemicals arrayed on a solid surface, by spotting, by synthesis in situ on the solid surface, or by depositing large beads onto the surface (e.g., from a split resin approach to combinatorial chemistry).

Still other aspects of the invention employ the CellSpot™ method to identify cells that can be immortalized to secrete multiply-specific immunoglobulins or immunospecific fragments thereof and to identify cells in general that secrete multiply-specific immunoglobulins or immunospecific fragments thereof. The CellSpot™ technology may also be used to identify cells that secrete immunoglobulins or immunospecific fragments of them that have desired, preferably human, glycosylation patterns.

### BRIEF DESCRIPTION OF THE DRAWINGS

FIG. 1A shows the results of measuring secretion level of single cells by measuring intensity and diameter of the footprint generated by the CellSpot™ method of the invention. FIG. 1B shows a comparison of these results with confirmatory data using macroscopic techniques on three orders of magnitude more cells.

FIG. 2 shows the distribution of secretion levels among individual cells in populations of a commercially available cell line and higher producing subclones thereof selected based on the size of their CellSpot™ intensity and diameter.

FIG. 3A shows the results of the invention method to select antibodies of high affinity, wherein the fraction of cells giving a detectable CellSpot™ declines as capture reagent density goes down, wherein that decline is more severe for weaker affinity clones. Alternatively, relative affinity can be estimated by normalizing for the amount of immunoglobulin in a CellSpot™ (since total signal is the product of intrinsic affinity/avidity and total Ig present); FIG. 3B shows a comparison of results based on this rank ordering method to an alternative commercially available method.

FIG. 4 is a diagram of the fragments used to generate antibodies against all exposed regions of a receptor protein, wherein the circled peptides represent those for which at least one specific antibody was identified.

FIG. 5 is a three-dimensional graph showing the number of antibody producing cells detected specific for each of the multiplicity of peptides prepared from fragments of a receptor protein in FIG. 4. Altogether, 2 million cells were screened against 9 probes concurrently.

FIG. 6 is a diagrammatic representation of an apparatus employed to conduct CellSpot™ analysis on bacterial cells, wherein the cells are supported on a large pore membrane (LP) which is positioned on a small pore (SP) membrane that provides a capture surface for proteins leaking from the periplasm, said small pore membrane positioned on a nutrient agar layer.

FIG. 7A is a low magnification image of the results of a CellSpot™ assay conducted with the apparatus of FIG. 6; FIG. 7B is a high magnification image of individual detection particles, imaged in one of two color channels.

FIG. 8A is a 2.5 times magnification and FIG. 8B is a 5 times magnification of anti-TI antibodies captured by cells displaying TI at their surface.

FIGS. 9A-9D show typical results from the binning technique described herein.

### MODES OF CARRYING OUT THE INVENTION

The invention will be described using antibodies or immunoglobulins for illustrative purposes. As is well understood in the art, the term “antibody” includes full length IgG and antibodies of other classes as well as single chain forms, e.g., camel antibodies and chicken antibodies. “Antibodies” encompass immunoreactive fragments such as Fab, engineered forms such as single chain Fv and the like. Chimeric antibodies, humanized antibodies and various permutations thereof are also invented in the definition. Thus, “antibodies” or “immunoglobulins” as used herein is a generic term referring to the various species that exhibit specific binding characteristics.

Although antibodies are used for illustration, the methods of the invention are not restricted to antibodies, and can be applied to any family of diverse binding agents, including recombinant proteins and peptides, or combinatorial chemistry libraries.

The present application describes a number of improvements in applications of the CellSpot™ assay described in WO 2005/045396. For convenience, the CellSpot™ method is described as follows, so that rather than repeating the steps common to all of the assays described herein, the shorthand term CellSpot™ method can simply be used.

In the CellSpot™ method, a capture surface is provided that permits the determination of the spatial location of positive or negative test results on a microscopic scale. Thus, the method includes microscopic examination of “spots” on the capture surface generated by the interaction of the surface with micro-reaction mixtures at discrete locations. The capture surface may be treated with capture reagent, or simple adsorption may be used. The CellSpot™ method is conducted so that the source of compounds or compositions to be detected is restricted to dimensions of ˜50-100 microns. In a preferred application of this method, the compounds to be detected are secreted proteins and the spatial arrangement is obtained by controlling the spatial arrangement of cells from which the proteins are secreted. The secreted proteins are often immunoglobulins, but the CellSpot™ assay is not limited to these. Any secreted protein, or peptide, may be employed in the CellSpot™ assay.

Preferred detection reagents in the methods of the invention are “multihued beads” which are described in detail in the above-cited WO 2005/045396 and in U.S. Pat. No. 6,642,062, incorporated herein by reference. Briefly, the multihued beads are particulates or “beads,” typically 50-1,000 nm in diameter, preferably in the range of 100-300 nm, composed of any material, but typically of latex or other polymers. Attached to the particulate support is a reagent specifically interactive with a desired analyte, such as the secreted protein, and a characterizing hue. The hue is obtained by providing the particulate with two or more signal generating moieties, wherein the signal from each is separately determinable, and the hue is determined by the ratio of the amounts of the signal generating moieties attached to the particle. Typically, the signal generating moieties are fluorophores which have distinctive emission maxima and can be separately determined. By varying the ratio of the fluorophores, a distinctive hue is obtained on the beads in each of a multiplicity of subpopulations. Thus, by use of such beads, each subpopulation having its own characteristic hue and specific binding reagent, a multiplicity of analytes may be simultaneously determined. Alternatively, detection of only a single analyte is possible.

The CellSpot™ method generates individual footprints of secreted protein(s) associated with individual cells. In order to identify an individual cell that has a desired level of secretion from among a large population of cells, one application of the invention method takes advantage of “binning”—i.e., examining simultaneously a multiplicity of individual secretion footprints. In this method, one or more cells, typically 1, 10 or 50 or more individual cells is added to a “bin,” typically the well of a microtiter plate, but generally any container with a base, typically flat, that can be assessed microscopically and of a diameter whereby individual footprints of 100-5,000 cells can be individually distinguished by the brightness of spots associated with their footprints after labeling with the multihued beads described above. The dimensions of the “bin” should be such that the entire base of the bin can be surveyed quickly, and such that the individual cell footprints can be distinguished. The originally added cells are then cultured to obtain a suitable population, typically 5-10 divisions or populations of several thousand cells, to obtain the desired test population. The cells will automatically settle to the base and the secreted footprint is captured at the base. If necessary, the base may be supplied with capture reagents suitable to the proteins to be assessed. For example, if antibody secretion is to be measured, a reagent such as Protein A that reacts with the constant region of immunoglobulins generally might be used. The cells are then removed from the bin and the footprints which remain are then assayed by labeling them with the multihued beads described above. In this way, bins that contain large numbers of cells that have exceptionally bright footprints can be used as the source of cells for further identification to obtain individual cells that have the desired level of secretion.

In one embodiment, a portion of the population of cells is removed before the footprints are assayed and this portion is then used (if it is determined from assessing the remainder that high producers are present) as a source for further testing either by limiting dilution or by plating on a membrane as individual cells or microcolonies for further identification of individual cells from among those in the remainder of the bin.

Once an individual cell that has a desired secretion level is identified, it too can be cultured and the resulting clonal population divided into suitable portions for replicate testing in the same CellSpot™ manner to verify the stability of the clonal population.

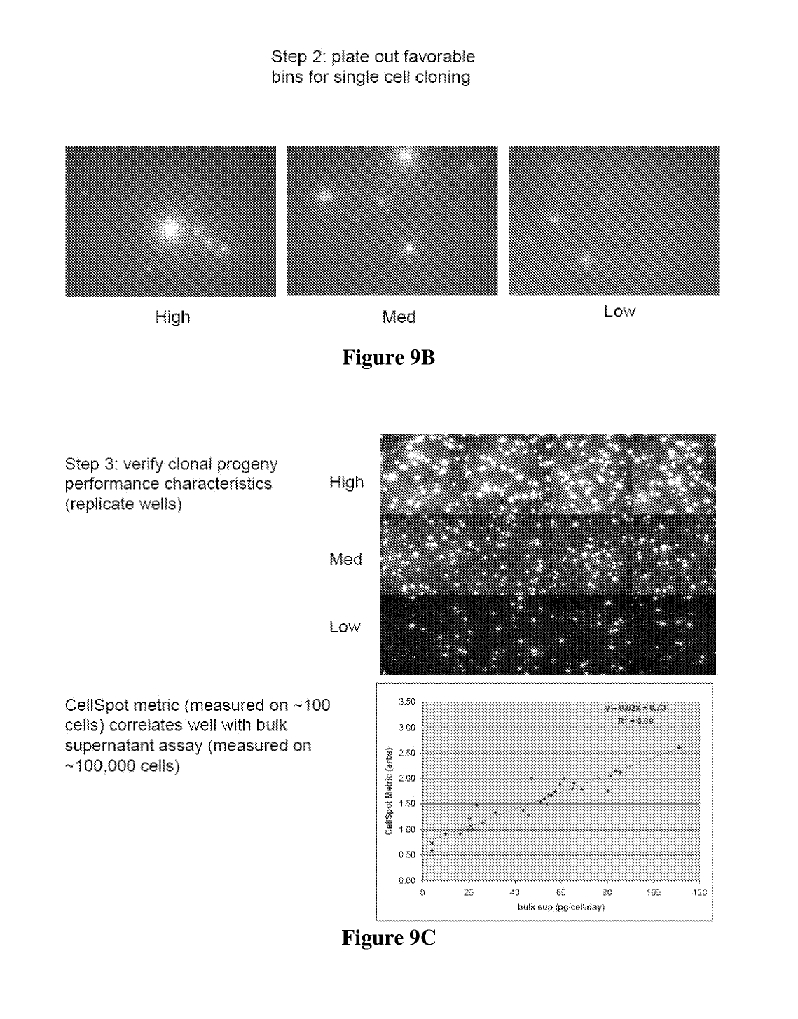

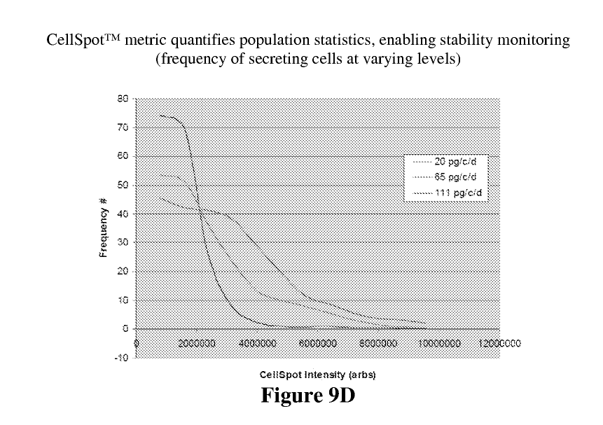

FIGS. 9A-9D show typical results from the simultaneous assay of multiple secreting cells in the foregoing binning technique. FIG. 9A shows a composite of results from various individual bins assayed as described above. It is clear that some of the bins contain a high proportion of cells with high secretion levels, while others are not so successful.

FIG. 9B shows the results when individual cells from the bins are placed on a supporting membrane and the footprints obtained from high, medium and low producing cells.

FIG. 9C shows the results obtained by assaying bins of clonal progeny of individually identified cells that have been cultured to obtain clonal populations. As seen, the high producing parent produces multiple high producing progeny that are consistent across replicates, whereas medium and low producers provide progeny that have similar patterns as the parental cell. The graph in FIG. 9C shows the correlation between the secretion levels measured on a collection of about 100 cells using the CellSpot™ assay with the results obtained using a bulk supernatant.

FIG. 9D shows the distribution of secretion levels among individual cells. Secretion levels obtained from bulk supernatants are shown in the box and these correlate well with the frequency with which high or low intensity cells are found within the population.

In some applications of the CellSpot™ technique, immunoglobulins or fragments thereof are particularly significant. For example, it is often desirable to identify single immunoglobulins that are able to bind more than one antigen. Such “multiply specific” antibodies may bind two or more, e.g., 3, 4 or even 5 different antigens. Such antibodies are particularly useful in therapeutic contexts as they expand the ability of the antibody to bind, for example, allelic variants of receptors or to related receptors such as HER2 and HER3. Such immunoglobulins may also bind multiple cytokines which may be helpful where more than one cytokine binds to the same receptor. For example, the cytokines CCL3, CCL5, CCL7 and CCL13 all bind to the CCR1 receptor and to one of the CCR2 and CCR5 receptors. Thus, the CCR1 receptor, for example, recognizes multiple cytokines and it would be desirable to find an antibody that has the same spectrum of binding. It is often desirable as well to bind to a discontinuous epitope, e.g., one formed from portions of both subunits of a heterodimer, such as an ion channel. It is also useful to provide antibodies that bind to the same epitope on homologous proteins from human and an animal model (e.g., primate or rodent) used in evaluating potential clinically applicable monoclonal antibodies. An antibody that recognizes the “same” protein in human and model permits toxicity and efficacy studies to be done in the animal model with the multiply specific antibody as a surrogate for the clinical candidate, or as the clinical candidate itself.

In the application of CellSpot™ to identifying cells that secrete such multiply-specific immunoglobulins, two basic approaches may be employed. In one approach, cells isolated from immunized models such as rodents, rabbits, or even human volunteers, are individually contacted with the particulate labels used in CellSpot™ wherein a multiplicity of labels containing a multiplicity of antigens is employed. It is then determined using the aid of a microscope the number of the multiple particulate labels associated with the cells. Cells associated with approximately equal numbers of more than one antigen-specific label are identified as cells that can be immortalized to secrete the desired immunoglobulins.

In an important embodiment of this aspect of the invention, each cell is supported on a membrane, optionally further containing a matrix that retains the cells, with secretion of the antibodies through the membrane to a capture surface, as is further described below.

It is known that the glycosylation pattern on immunoglobulins affects both their efficacy in cell killing (ADCC) and their pharmacokinetics. Therefore, for example, in preparing antibodies for human therapeutic use, it is important to assure that the glycosylation pattern of these antibodies is as close as possible to human patterns. In particular, the inclusion of fucose in glycosylation moieties in human antibodies is undesirable. In one aspect of the invention, cells that secrete antibodies with appropriate glycosylation patterns can be identified using the CellSpot™ assay. Because it is possible to detect easily up to 20-50 individual particulate labels at a location on a capture surface or associated with a single cell, particulate labels containing lectins that bind individual carbohydrate moieties can be used to identify these cells. A multiplicity of such lectins is indeed commercially available, for example, from Qiagen where the lectins are arrayed on a microscope slide.

In the method of the present invention, the individual lectins are associated with particulate labels of different hues and these labeled lectins used to assess the secreted antibodies. The foregoing method is appropriately applied to recombinant cell lines that secrete antibodies of desired specificities which are often non-human cell lines. Mutagenesis may be necessary to provide individual cells that can then be identified as secreting antibodies with appropriate glycosylation. Retention of the desired glycosylation pattern can also be readily monitored during scale up of the cell line for use in fermentors (expansion of >10¹²-fold is common, allowing many opportunities for loss of the favorable phenotype).

Thus, in a second embodiment, cells that secrete desired antibodies are supported on a membrane which permits the immunoglobulins secreted to pass through the membrane to a capture surface. The capture surface may, if desired, comprise non-specific immunoglobulin capture reagents. The location of the antibodies on the capture surface corresponds to the location of the secreted cell on the membrane. The capture surface is then probed with a multiplicity of lectin-containing particulate labels of various hues corresponding to the variety of lectins coupled to them. The pattern of labeled lectins associated with each secreted antibody can then readily be determined. Typically, the collection of labeled lectins will contain lectins that bind both desired and undesired sugars. Since only five or six different lectins are needed to approximate a satisfactory glycosylation pattern, the detection resolution is well within what is needed for this purpose. Those antibodies associated with lectins that bind desired, but not undesired carbohydrate moieties are then selected at a location on the surface which is then correlated with the appropriately mutagenized cell. This cell can then be propagated for production of antibodies with desired glycosylation.

A similar system is used to identify cells, typically, but not exclusively, recombinant cells or hybridomas or otherwise immortalized cells that secrete antibodies with multiple specificity. A similar format is employed wherein the cells are supported individually or in microcolonies on a membrane that permits passage of the secreted immunoglobulins or fragments to a capture surface. In this case, the particulate labels contain a multiplicity of antigens or epitopes, each associated with a particular hue generated by the particulate label. Locations on the membrane where a multiplicity of such labels is detected are identified as associated with cells that secrete multiply-specific immunoglobulins or fragments. Thus, antibodies that bind two, three, four, five or more antigens or epitopes can be identified.

The various aspects of the invention include specifically:
A method to obtain antibodies immunoreactive with a functional region of a protein, which method comprises
   fragmenting the protein into at least 5 fragments;
   coupling each of said fragments to an immunogenicity enhancing component;
   immunizing one or more subjects with each said coupled fragment;
   harvesting antibody-producing cells from the subject(s);
   testing individual harvested cells for antibodies that are immunoreactive with each immunizing fragment and with the intact protein, but not immunoreactive with the remaining fragments;
   selecting cells producing such antibodies; and
   testing the antibodies secreted by said cells for their effect on the function of the protein.
A method to detect the presence or absence of at least one protein secreted by bacterial cells which method comprises
   incubating a multiplicity of microcolonies derived from single cells on a porous membrane comprising pores that permit transit of small molecules and proteins, but do not permit transit of bacterial cells
   under conditions, wherein said at least one protein is secreted;
   permitting any secreted proteins to transit the pores onto a capture surface placed below said porous membrane;
   said capture surface optionally having been treated with a capture reagent that binds at least one desired protein;
   removing the porous membrane,
   treating the capture surface with particulate labels coupled to a reagent reactive with the at least one secreted protein;
   removing unbound labels; and
   detecting microscopically the presence or absence of any bound label as demonstrating the presence or absence of said at least one secreted protein.
An improved method of conducting a CellSpot™ assay, wherein said improvement is selected from the group consisting of
   a) use of a microplate carrier that holds a membrane on which cells are positioned for the assay which comprises a grid pasted to the bottom thereof; style
   b) use of a membrane on which cells are positioned for the assay which comprises scattered fluorescent particles of 5-10μ diameter;
   c) use of Mebiol™ gel as an immobilization medium for cells on a membrane on which cells are positioned for the assay;
   d) use of a means for sliding a stage holding the membrane on which cells are positioned for the assay laterally from under a microscope to permit vertical access by pipette.
A method to evaluate the effect of the composition of medium on secretion levels, which method comprises observing the secretion level of individual cells or microcolonies in the presence of said medium using a CellSpot™ assay, and
   comparing said level to that obtained and measured by the same assay in the presence of a medium of a different composition.
A method to monitor the duration and amount of protein secreted by a single cell which method comprises conducting a CellSpot™ assay with respect to each cell as a function of time.
A method to measure the ability of a substance to undergo endocytosis which method comprises
   providing a test substance coupled to a DNA intercalating dye;
   treating one or more cells with said labeled test substance; and
   detecting the presence, absence or amount of said DNA intercalating dye in the nucleus of said cell.

In one embodiment, a multiplicity of substances each labeled with a different intercalating dye is used to treat said cells.

The following examples are offered to illustrate, but not to limit the invention.

### EXAMPLE 1

### Determination of Secretion Level

Hybridoma cells that secrete immunoglobulins were obtained from ATCC and deposited onto a membrane with 0.4 μm pores in contact with an underlying polystyrene surface coated with anti-immunoglobulin. The cells were suspended in 1.2% methylcellulose and to secure the cells, the plate was centrifuged briefly. The secreted IgG passes through the membrane onto the coated polystyrene surface. The membrane containing the cells was supported on a plastic holder that permits it to be removed from the capture surface; the holder was a modified Transwell® material obtained from Costar®, for which a special holder was designed that brings the membrane into contact with the capture surface.

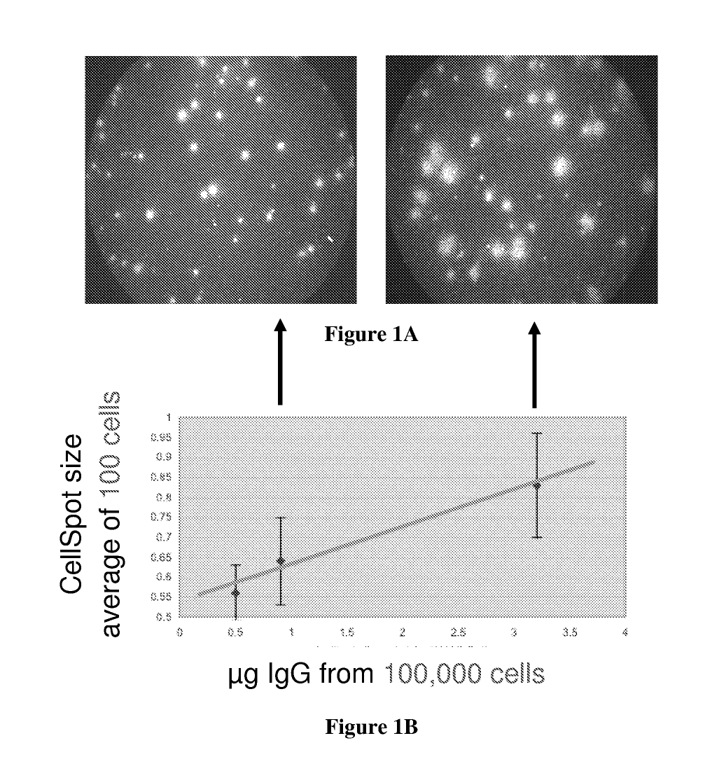

After incubation for 2 hours, the membrane was removed and the underlying polystyrene surface incubated with detection particles, washed, and then scanned with both a low and high magnification microscope. The results are shown in FIG. 1A which shows the contrasting patterns of cells with high and low secretion levels. Each “spot” represents a single cell in each case. The intensity and diameter of the spot was quantified and used to construct a metric of secretion. These secretion levels were correlated with independently measured secretion level from macroscopic supernatant samples of the hybridoma cells that were used to obtain the footprints shown in FIG. 1A. There is good correlation between the two metrics, as shown in FIG. 1B.

This method may be applied to a library of transfected cells, wherein the site of integration of the coding DNA into the chromosome influences the ultimate secretion level. A large number of randomly integration events can thus be surveyed efficiently.

### EXAMPLE 2

### Selection of High Secretion Clones

The cell line ATCC 60525 was separated into 10,000 individual cell assays using the method of Example 1. Three individual cells were picked and cultured as subclones. The subclones were again subjected to the CellSpot™ assay of Example 1 wherein 1,000 cells were assayed for each subclone.

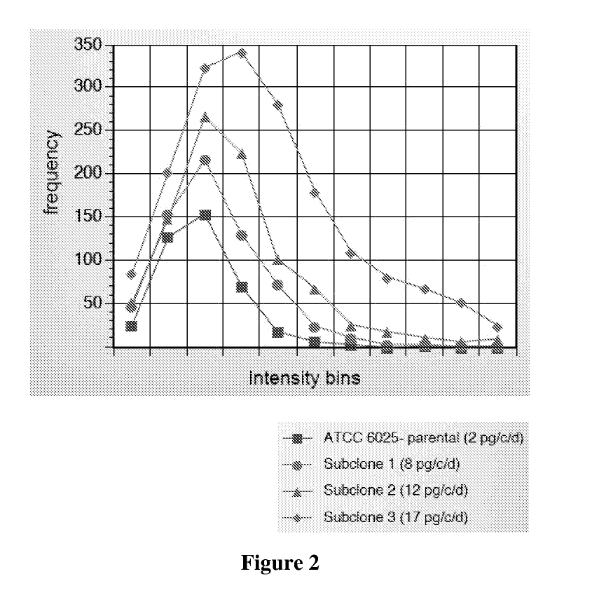

As shown in FIG. 2, the distribution of secretion levels is shifted to higher secretion levels for the members of the three selected subclone parents resulting in an overall improvement of nine-fold for the highest secretor, as measured by macroscopic supernatant assay.

The same methodology is applicable to any population of cells that vary in their secretion level, for example a library of transformed CHO cells. Depending on where the DNA for the secreted protein integrates in the genome, expression level will vary. For more reliable identification of high secretors, the cells are allowed to divide in “bins” of 100 parental cells per well of a standard 96 well microplate. CellSpot™ footprints are analyzed after transfer of the cells to a duplicate plate. Those wells with a multiplicity of high secreting cells, presumably derived from one parental cell, are then plated out in the modified Transwell and single cells picked based on their secretion level as determined by analysis of the resulting CellSpots.

A large library of random insertion sites can be readily screened in this manner. The chromosomal integration site for an unusually high secreting clone can be determined by DNA sequencing of the insert gene and its flanking DNA. Directed insertion of the gene for a new expressed protein into that site can then by accomplished using site specific recombination. If the transfected gene contains recognition sequences for a site-specific recombinase, such as the Cre-Lox or frt system, the expressed gene can be excised, leaving behind the recognition sequences that can be exploited in future transfections.

### EXAMPLE 3

### Determination of Affinity

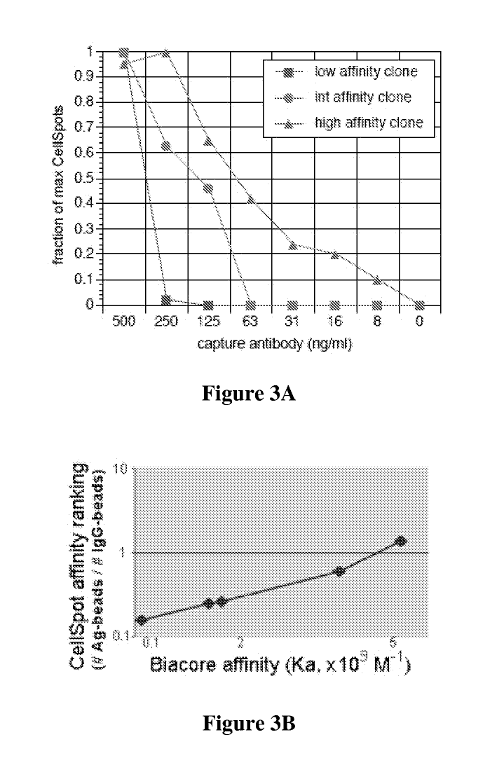

Three hybridoma cell lines were determined to secrete antibodies of varying affinity for the same antigen by the Biacore™ commercial instrument method. Each cell line was assayed as set forth in Example 1 using varying concentrations of capture antibody on the capture surface. The clones differed in the frequency of input cells yielding detectable antibodies according to their predetermined affinity as shown in FIG. 3A.

The assay was conducted by placing a fixed concentration of capture antibody on the surface and counting the number of spots observed at high surface antibody concentration, and assigning a value of 1.00 to that number of spots (100%), as shown on the Y axis of the graph in FIG. 3A. The capture antibody on the surface was then progressively diluted in replicate wells, and the number of spots observed at each dilution. The ratio of this number to that observed at the concentration assigned the value of 1.00 was then plotted on the Y axis of FIG. 3A.

As indicated, in the clone of low affinity, spots were detected only at a concentration of capture antibody at >250 ng/ml. For an intermediate affinity clone, spots could be detected at concentrations above 63 ng/ml for the coating with capture antibody, and for the high affinity clone the number of spots did not decay to zero until the capture antibody concentration plated at the surfaces fell below 8 ng/ml. The reduced level of spots formed as capture reagent density declines reflects a decrease in the avidity effect.

FIG. 3B shows a different approach to rank ordering clones by affinity. In this instance, the CellSpots were probed with both antigen conjugated beads and with beads conjugated to an anti-immunoglobulin. Since raw signal (number of antigen beads bound per CellSpot™) is proportional to both amount of secreted antibody captured and the intrinsic affinity (or avidity) of the antibody for antigen, the ratio of antigen beads to anti-Ig beads provides a normalization for the abundance of captured antibody. A comparison to standard Biacore™ affinity assay results is shown in FIG. 3B, with the good correlation establishing the ratio metric as a reliable guide to relative affinity.

### EXAMPLE 4

### Preparation of Antibodies for Fragments of a Membrane Receptor

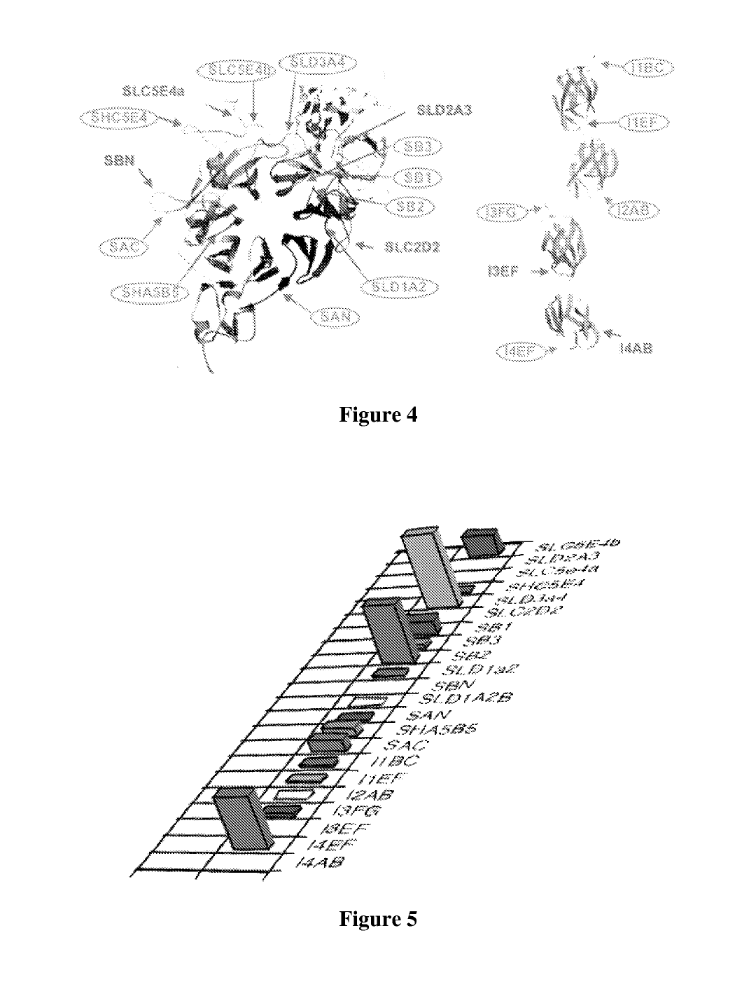

FIG. 4 shows a diagram of the extracellular domains of a receptor protein and the location of fragments used for generation of antibodies. The indicated regions were coupled to immunogen (KLH) and used to immunize mice. Spleen cells were harvested and assayed individually according to the CellSpot™ technique of Example 1. In the case of almost every peptide, at least one cell was observed to secrete antibodies that reacted with the immunizing peptide. For 70% of the peptides (16 of 22), these antibodies were specific for the immunizing peptide as compared to peptides from nearby on the receptor. FIG. 5 displays as bar height the frequency of cells secreting antibodies that met three criteria which indicate specificity for the immunizing fragment: the antibody binds only to the fragment used as an immunogen, the antibody binds to the intact protein, and the antibody does not bind to a related intact protein. For some of the peptides, many cells secreted antibody meeting these criteria, but for others, only a single cell was identified, out of ˜2 million total cells screened. In this manner, the functional utility of antibodies targeting different regions of the protein can be assessed, even if different regions vary markedly in their immunogenicity.

### EXAMPLE 5

### Integral Membrane Protein Antigen

Cells expressing an integral membrane protein, TR1, fused at its intracellular terminus to a hemagglutinin tag, were grown in standard media. Approximately 5-10 million cells were solubilized in tris-buffered saline with detergent for 30 minutes. Suitable detergents include CHAPS as a preferred choice, n-octyl-β-D-glucopyranoside, n-decyl-β-D-mannopyranoside, and n-dodecyl-β-D-maltopyranoside. Solubilization was confirmed by Western blots, using a first generation antibody to TR1. Rabbit polyclonal antibody against the tag was covalently attached to fluorescent particles using Schiff base chemistry. After solubilization, insoluble material was removed by centrifugation. Beads conjugated to an irrelevant antibody (anti-hIgG) were added to the supernatant for 20 min, then centrifuged to remove non-specifically binding material. The supernatant was mixed with 40 μl of the anti-HA beads and incubated at 4° C. for 4 hours with gentle mixing. These beads were centrifuged and washed 3 times with solubilization buffer. The beads were then resuspended in solubilization buffer and used as probes in the CellSpot™ as described in Example 1. Positive signal was seen with hybridoma cells secreting the first generation anti-TR1 antibody, but not with a control hybridoma line.

### EXAMPLE 6

### Secretion Footprint from Bacteria

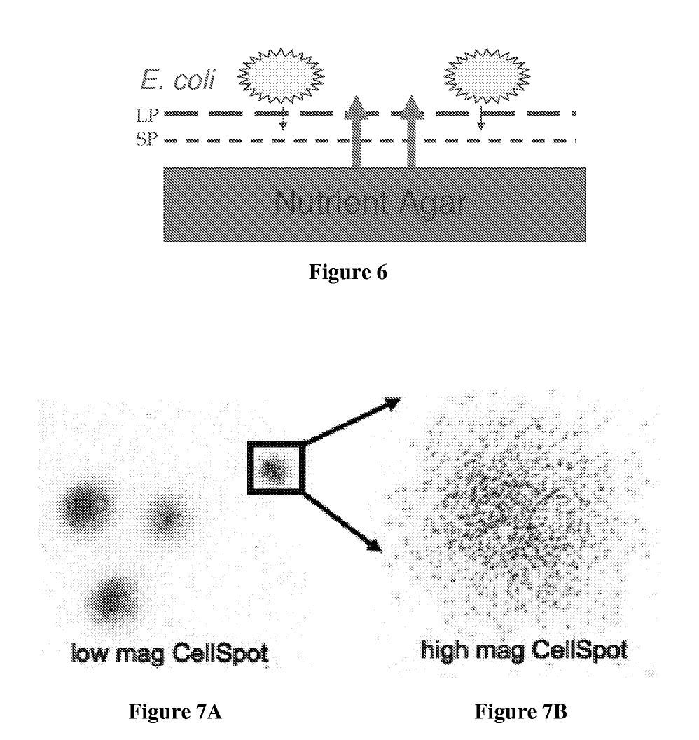

FIG. 6 is a diagram of the apparatus used in this example for characterizing genetically modified E. coli with respect to their secreted immunoglobulins. As shown, the cells are positioned microcolonies on a nitrocellulose membrane where they will grow into small colonies.

This top membrane is placed above a capture surface which is constructed of a flat plastic membrane a few micrometers thick with well defined holes, e.g., drilled by nuclear pore etching. In the “nucleopore” process, small holes are made by irradiation and then expanded by chemical etching. The capture surface in this example is polyester with holes of 500 nm diameter covering 1% of the surface. The capture membrane may also be derivatized with a carboxy-dextran layer to provide more sites for immobilizing a capture reagent. Ig-secreting cells are then analyzed as shown in FIG. 7A, in which the top membrane containing bacteria is positioned on a capture membrane which in turn is positioned on a bed of nutrient agar. The capture antibody attached to the capture membrane is an anti-immunoglobulin antibody; beads are labeled with a specific antigen and used to probe the CellSpots created on the capture membrane. As shown in FIG. 7B, individual micro-colonies give robust CellSpots, which can be examined at high magnification in each color channel for determination of bead types bound, extending the CellSpot™ assay from mammalian cells to bacterial cells.

It has thus been demonstrated that there is sufficient leakage of recombinantly produced immunoglobulin from the periplasmic space for ready detection; thus, the cells do not need to be subjected to osmotic pressure in order to release sufficient immunoglobulin to detect.

This system is particularly useful for screening randomly constructed immunoglobulin libraries. In such an application, an E. coli culture is transfected with expression plasmids for 100 different heavy chains and 100 different light chains, using two selectable markers on the vectors to select for cells expressing both a heavy and light chain. The secreted antibodies are then analyzed as set forth above. The same method can be applied to any recombinant library of proteins.

### EXAMPLE 7

### Viral Plaque Assay at Cellular Resolution

Cells suspected to, or known to be infected by virus are spread, optionally on a membrane, to effect capture of released substances on a capture surface, as done in the CellSpot™ format. In this case, the capture surface is provided with antibodies specific for viral proteins. The virus particles, released from the cells, either by lysis or budding, are then captured in the region of the cells and labeled with particulate carriers of individual hues. Multiple capture antibodies may be used to provide increased reliability of detection and classification, for example, with regard to strain type. Viruses released from only a single cell is detectable. A large lawn of cells can readily be screened by this method.

### EXAMPLE 8

### Identifying Highly Secreting Cells by Binning

A sample of 20 immortalized antibody-secreting cells is placed in the well of a microtiter plate and cultured to a population of 2,000 cells. One-half of the culture is then removed and set aside and the remaining 1,000 cells allowed to settle and secrete antibodies onto the base of the well which has been provided with a coating of protein A to capture the antibodies. The cells are then washed away and the footprints of secreted antibodies are interrogated using multihued beads coupled to antigen immunoreactive with the desired antibodies. The multihued beads are labeled with fluorophores and detected in a wide field detection microscope as individual footprints. The bin is then assessed for the presence of a substantial number of brightly fluorescing footprints.

The removed portions of those bins that contain substantial numbers of brightly fluorescing footprints are then used as a source for further assessment of individual cells. The cells in the portion of culture removed are then tested in the CellSpot™ assay by placement on a membrane to assess individual footprints from which individual cells can be recovered.

Maintenance of high secretion levels is then assured by culturing the recovered high secreting cells and performing replicate determinations using the binning technology on their progeny populations.

### EXAMPLE 9

### Antibody Capture on Indicator Cell Layer

A monolayer of live 3T12-TI-fibroblasts which display TI protein at their surface is prepared as a cell capture surface.

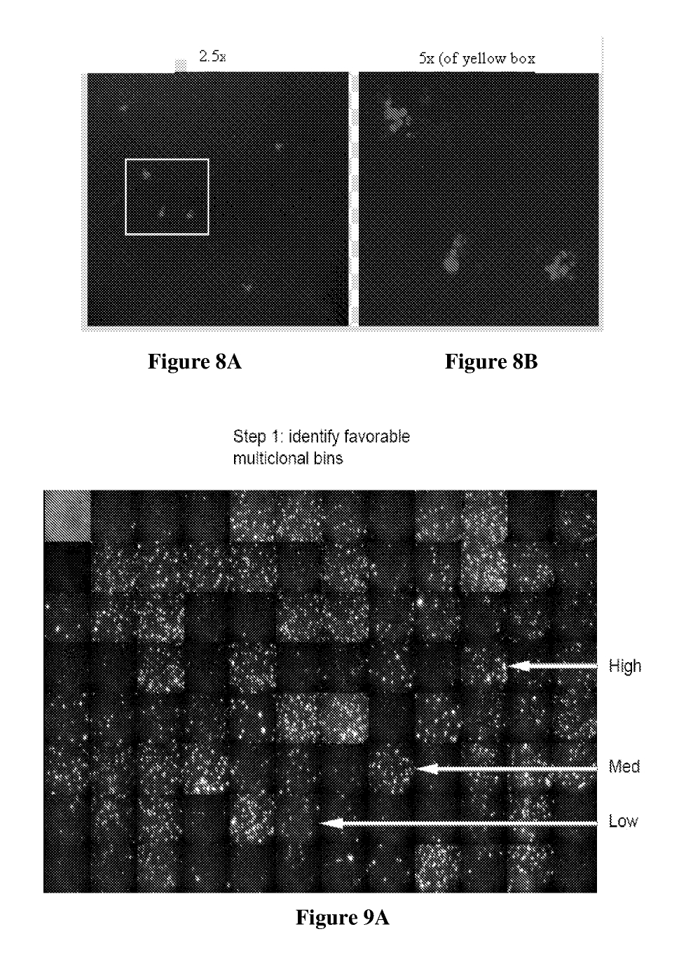

Immortalized spleen cells derived from a mouse immunized with TI are then placed on a membrane overlying the surface and secretion is then permitted to occur. The membrane is then removed and the TI-fibroblast capture cells are fixed and stained for the captured antibody with a fluorescence-tagged anti-Ig antibody. Fixation also exposes internal antigens, so, for example, intracellular phosphorylation could be detected. Typical results are shown in FIGS. 8A and 8B at 2.5× and 5× magnification. As shown, the cells displaying TI form a successful capture surface reagent.

### EXAMPLE 10

### Antibody Internalization Assay

It is sometimes useful to generate an antibody that stimulates uptake of the antibody and associated proteins into the cell via endocytosis. For example, such internalization may reduce the quantity of detrimental protein at the cell surface, or it may be useful for delivery of a drug into the interior of the cell. Association of the antibody with a DNA intercalating dye provides a sensitive measure of internalization of the complex since the dye only becomes fluorescent upon interaction with cellular DNA. A library of candidate targeting antibodies is fused to a dye capture domain (e.g., avidin to bind biotin-dye conjugate, or an albumin binding protein to bind an albumin bound dye). Cells expressing the candidates are exposed to a surface providing an indicator cell layer in the presence of the optionally derivatized dye, which binds to the dye capture domain of the secreted antibody. Uptake into the indicator cells is assessed by nuclear fluorescence when the intercalated dye is bound to DNA.

### EXAMPLE 11

### Alternative Scaffolds

Antibodies are not the only diverse population of binding agents. Other protein families also include readily modifiable loops analogous to the complementarity determining region of antibodies. A specific example is glutathione transferase. Mutating a specific loop results in a randomized library of “glubodies”, whose members display considerable variety in binding profiles for small molecule ligands, as disclosed in Napolitano, et al., Chem. Biol. (1996) 3(5):359-367).

In addition to recombinant proteins, the invention can be applied to small recombinant peptides. As described in WO 01/81375, avian pancreatic peptide (aPP) is a 36 amino acid long peptide that folds into a rigid structure, with a melting temperature of 65° C. Variation of the solvent exposed residues does not significantly affect the stability of the folded peptide. Fusing aPP to a tether, e.g., the Fc region of an antibody, facilitates screening of a randomized library of aPP variants using the CellSpot™ methodology.

More generally, any array of ligands can be screened by the CellSpot™ multiplexed analysis technique. For example, a combinatorial chemistry library can be synthesized on a planar surface, as described for example in U.S. Pat. No. 5,744,305. In this method, photolithography is used to create binary masks for controlling release of light sensitive protecting groups. Using the CellSpot™ approach to increase sensitivity of detection, the spot size can be reduced. Further the specificity of the ligands for a family of target proteins can be assessed. Alternatively, the compounds can be synthesized on beads, with a cleavable linker. Release of the compound from the beads and capture on a surface thereby generates a distribution of binding partners that can be probed in the same manner as a distribution of antibodies. Rather than recovering the cell that produced the antibody, the bead that produced the compound is recovered.

1. A method to identify antibodies immunoreactive with a functional region of a protein, which method comprises testing the effect on said function of antibodies secreted by each of cells resulting from immunization by each of at least 5 fragments of said protein, wherein said cells are obtained by
fragmenting the protein into at least 5 fragments;
coupling each of said fragments to an immunogenicity enhancing component;
immunizing one or more subjects with each said coupled fragment;
harvesting antibody-producing cells from the subject(s);
testing individual harvested cells for antibodies immunoreactive with each said fragment and with the intact protein, but not immunoreactive with the remaining fragments; and
selecting cells producing said antibodies, and optionally testing the antibodies secreted by said cells for their effect on the function of the protein.

2. A method to detect the presence or absence of at least one secreted protein from bacterial cells, which method comprises
microscopically observing the presence or absence of particulate label coupled to reagent specific for said protein as demonstrating the presence or absence on the surface of a capture surface optionally containing a capture reagent that binds the protein; and
wherein the capture surface has been placed beneath a porous membrane upon which bacterial cells are supported as microcolonies grown from a single cell, said membrane comprising pores that permit transit of small molecules and proteins but do not permit transit of bacterial cells.

3. The method of claim 2, wherein said capture surface comprises a nuclear-etched membrane surface, optionally derivatized with a hydrogel to which capture reagent is attached.

4. The method of claim 2, wherein the at least one secreted protein is an antibody or fragment thereof produced by bacteria modified to express at least the variable region of a light chain and at least the variable region of a heavy chain.

5. The method of claim 4, which employs a multiplicity of microcolonies each resulting from a single cell modified to express said variable regions, said single cells resulting from treating a culture of bacterial cells with nucleotide sequences that, when transfected into said bacteria produce at least 10 different light chain variable regions and at least 10 different heavy chain variable regions.

6. A method to employ epitopes of a membrane-bound protein as detection reagents in CellSpot™ assays, which method comprises
expressing said protein optionally in host cells, said protein comprising an intracellular region which contains a binding partner to a complementary moiety;
disrupting any said host cells; and
coupling said protein to a particulate label by interaction between said binding partner and its complementary moiety which complementary moiety is associated with a particulate label.

7. The method of claim 6, wherein said protein is produced in a cell-free system and recovered in the presence of detergents.

8. The method of claim 6, wherein said binding partner is heterologous to the membrane-bound protein.

9. The method of claim 6, wherein the binding partner is a histidine tag, a FLAG epitope, or an enzyme complementary to a suicide substrate.

10. A method to employ a membrane-bound protein as a detection reagent for secreted proteins, which method comprises
preparing a capture surface comprising cells that produce the membrane-bound protein at a desired level;
treating the surface with secreted protein to be detected; and
detecting any secreted protein that interacts with cells that produce the membrane-bound protein at said level,
whereby secreted protein that interacts with cells producing said protein at said level is identified as secreted protein that interacts with the membrane-bound protein,
wherein said secreted protein does not interact with, or interacts at a lower amount with any cells, if present, not so producing said protein.

11. The method of claim 10, wherein said interaction is binding, or comprises intracellular signaling upon exposure of intracellular antigens by fixation and staining.

12. The method of claim 10, wherein a second cell type is included in the capture surface, said second cell type expressing little or none of the membrane bound protein, said second cell type being distinguishable from the first cell type.

13. A method to immortalize human peripheral blood cells for application to assay methods that require 20 or fewer cells, which method comprises infecting said cells with Epstein Barr virus and harvesting the cells after 20 or fewer cell progeny are obtained.

14. The method of claim 13, wherein said assay method is a CellSpot™ method.

15. A method to identify a protein with high affinity for its binding partner which method comprises treating a series of capture surfaces with said protein, wherein said series of capture surfaces contains a binding partner for said protein at a series of diminishing concentrations on said surface;
detecting the binding of protein to each of said surfaces, whereby a protein that continues to bind said surface at low concentrations of binding partner is identified as a protein with high affinity for said binding partner.

16. A method to identify cells with desired secretion levels and/or desired specificity of a secreted protein which method comprises
plating a multiplicity of individual single cells or of individual microcolonies, optionally supported on a porous membrane;
allowing secreted proteins from said cells to contact an underlying capture surface placed under the membrane, when present, wherein said secreted protein is captured on the capture surface;
removing the membrane, if present, to expose the capture surface;
removing unbound proteins from the capture surface;
treating the capture surface with one or more labels at least one label comprising a binding partner specific for said protein and further comprising a signaling moiety;
examining the capture surface microscopically to determine the size and/or intensity and/or nature of the signal emitted by the label;
whereby larger or more intense areas of signaling indicate cells having a high level of secretion for said protein, and
predominance of signal associated with label specific for said protein indicates cells secreting protein of desired specificity.

17. The method of claim 16 wherein the protein is contained on a virus infecting said cell, thereby permitting determining a fraction of cells infected by the virus.

18. A method to identify an insertion site into which insertion of DNA coding for a secreted protein provides a high expression level of a desired protein, which method comprises
inserting a nucleotide sequence encoding the desired protein into a multiplicity of insertion sites in the DNA of a population of cells or of microcolonies; and
individually evaluating secretion rates of the encoded protein of each transfected cell or microcolony; and
correlating the level of secretion of the protein with its cell or microcolony of origin, thus identifying cells or microcolonies which provide high levels of secretion, and thus permitting identification of the insertion site.

19. A method to identify individual cells with desired levels of secretion of at least one protein which method comprises
(a) culturing one or more cells in a bin to expand the cell population to a desired population level;
(b) optionally removing a portion of said culture;
(c) allowing the cells to settle to the bottom of the bin;
(d) allowing sufficient time for the cells to secrete protein(s);
(e) removing said cells from the bin, leaving behind secreted protein(s) as a footprint of each individual cell; and
(f) labeling said footprints to determine the amount of protein(s) in each footprint;
thereby identifying a bin that contains individual cells that secrete protein(s) at a desired level, and identifying individual cells that secrete protein(s) at a desired level.

20. The method of claim 19, wherein each label is supplied as a particulate comprising one or more fluorophores and a detecting reagent that binds a specific protein.

21. The method of claim 20, wherein a multiplicity of secreted proteins is labeled by supplying a multiplicity of subpopulations of particulate labels, each subpopulation comprising a different detecting reagent and a different ratio of fluorophores coupled to the particulates.

22. The method of claim 19, which comprises removing a portion of the culture in step (b) and assessing the ability of said cells to secrete high levels of protein by testing each cell by a method which comprises plating individual single cells from the removed portion onto a porous membrane;
allowing secreted proteins from said cells to contact an underlying capture surface placed under the membrane, wherein said secreted protein is captured on the capture surface;
removing the membrane, to expose the capture surface;
removing unbound proteins from the capture surface;
treating the capture surface with a label comprising a binding partner for said protein and a signaling moiety;
examining the capture surface microscopically to determine the size or intensity of the signal emitted by the label;
whereby larger or more intense areas of signaling indicate cells having a high level of release for said protein.

23. The method of claim 22, which further includes culturing individual cells to obtain a desired population;
dividing said population into replicate samples; and
testing said samples by
culturing one or more cells to expand the cells to a desired level of progeny;
placing replicate samples of the progeny into bins;
allowing the progeny cells to settle to the bottom of the bins;
allowing sufficient time for the progeny cells to secrete any protein(s);
removing said progeny from the bins, leaving behind secreted protein(s) as footprints of each individual cell; and
labeling said footprints to determine the amount(s) of protein(s) in each footprint;
wherein identifying bins that retain cells that secrete protein(s) at a desired level, confirms that the progeny continue to secrete said protein(s).

24. A method to analyze a combinatorial library for ability to bind one or more binding partners, which method comprises
displaying each member of the combinatorial library at a specific location on a capture surface;
treating said capture surface with a labeled form of at least binding partner against which the members of the library are to be tested; and
at each location, detecting the presence or absence of the labeled binding partner using microscope detection,
wherein the label is a multihued bead, or
providing each member of the combinatorial library with a distinctive label;
providing a capture surface containing the desired binding partner; and
treating said capture surface with a mixture of the members of said library; and
detecting any labeled bound members microscopically.

25. The method of claim 24, wherein said treating is with a multiplicity of binding partners each bearing a distinctive label.

26. The method of claim 25, wherein the members of the combinatorial library are secreted proteins.

27. A method to identify cells that can be immortalized to secrete a multiply-specific immunoglobulin, which method comprises
testing individual B-cells derived from spleen, lymph nodes, mucosal-associated lymphatic tissue or peripheral blood, or other cells that express antibody or antibody-like binding agents, for secretion of antibody that binds to two or more different antigens by treating each said B-cell or antibodies secreted by said B-cell with a first particulate label comprising a first antigen, a second particulate label comprising a second antigen different from the first and optionally additional particulate labels comprising additional antigens different from the first and second antigens; and
determining microscopically the number of said first, second and any additional particulate labels associated with said cell,
whereby cells associated with approximately equal numbers of said first, second and any additional labels are identified as cells that can be immortalized to secrete said immunoglobulin.

28. The method of claim 27, wherein each said cell is supported on a membrane and any secreted antibodies are collected at a sample surface below said membrane.

29. The method of claim 28, wherein said membrane further contains a matrix to secure the cell to the membrane.

30. The method of claim 29, wherein said matrix contains the particulate label that binds to immunoglobulins in an antigen and epitope independent manner.

31. The method of claim 30, which further includes immortalizing the antibody producing cells prior to assay or after identified as secreting desired immunoglobulins.

32. A method to identify cells that secrete a multiply-specific immunoglobulin or a multiply immunospecific fragment thereof which method comprises
providing cells on a membrane, said membrane being permeable to secreted immunoglobulins and said membrane overlying a sample surface optionally comprising a capture reagent for immunoglobulins;
removing the membrane containing the cells; and
probing the sample surface with a multiplicity of antigens each labeled with a distinguishable particulate label; and
selecting a location on the surface which binds to two or more different antigens; and
correlating the selected location on the surface thus identified with the location of cells on the membrane,
thereby identifying cells that secrete a multiply-specific immunoglobulin or a multiply immunospecific fragment thereof.

33. A method to identify cells that secrete an immunoglobulin or fragment of an immunoglobulin having desired glycosylation which method comprises
providing cells in a format that allows capture of the secreted antibody on a sample surface optionally comprising a capture reagent for immunoglobulins; and
probing the sample surface with a multiplicity of lectins, some of which bind to desired glycosylation and some of which bind to undesired glycosylation, each labeled with a distinguishable particulate label; and
selecting cells whose secreted antibodies bind to lectins reactive with desired glycosylation but not to lectins reactive with undesired glycosylation;
thereby identifying cells that secrete an immunoglobulin or fragment of an immunoglobulin having desired glycosylation.
